(19) United States
(12) Patent Application Publication
(10) Pub. No.: US 2017/0042630 A1
(43) Pub. Date: Feb. 16, 2017

(54) Protective Hood for a Surgical Operation Field

(71) Applicant: Söring GmbH, Quickborn (DE)

(72) Inventors: Florian Neumann, Hamburg (DE); Frederick Klaus, Quickborn (DE)

(21) Appl. No.: 15/305,818

(22) PCT Filed: Mar. 19, 2015
(86) PCT No.: PCT/EP2015/055816

§ 371 Date: Oct. 21, 2016

(30) Foreign Application Priority Data
Apr. 29, 2014 (DE) 102014208074.6

(51) Int. Cl. A61B 90/00; A61B 17/32; A61B 90/40

(52) U.S. Cl. CPC A61B 90/05; A61B 90/40; A61B 17/320068; A61B 2017/1602

(57) ABSTRACT

A protective hood for a surgical operation field comprises a partition which separates a protection area from an outer chamber, and a protective surface which surrounds the operation field. The partition comprises a through opening which is surrounded by a sealing surface for a shaft of a surgical instrument. The claimed protective hood enables surgical treatment steps to be carried out without aerosols, vapour and similar being dispensed to the environment.

### BACKGROUND

The invention relates to a protective hood for a surgical operation field. The protective hood comprises a partition, which separates a protection chamber from an exterior, and a support surface surrounding the operation field.

In surgical treatment steps, tissue material is regularly ablated from the body of the patient. Various mechanisms can have the effect that the ablated tissue material does not remain in the operation field but instead spreads into the environment. This may be the case, for example, if the tissue material sprays into the environment along with an irrigation liquid. It is likewise possible that the tissue material is broken into very small particles in the surgical treatment step, with the result that the material can spread in the form of an aerosol into the environment. This problem arises in particular if the treatment step is carried out with a rapidly rotating surgical instrument, for example a drill or a milling cutter, or with an instrument that generates cavitation, for example an ultrasonic surgical instrument. From the point of view of hygiene, it is undesirable if tissue material escapes from the operation field and spreads into the environment.

### SUMMARY

A protective hood is provided for a surgical operation field, counteracting the spread of tissue material during a surgical treatment step

The partition has a through-opening which is surrounded by a sealing surface for a shaft of a surgical instrument.

Some terms will first be explained. The protective hood is intended to be placed with the support surface onto a surface area surrounding the operation field. In this state, a protection chamber forms which is delimited, on the one hand, by the operation field or the surrounding area of the operation field and, on the other hand, by the partition. If the protective hood lies separate, then the protection chamber transitions into the environment via an opening surrounded by the support surface. The support surface is designed to encircle the operation field when the protective hood is used as intended.

By way of the through-opening, the protection chamber can be accessed by a surgical instrument. The surgical instrument is guided through the through-opening, such that the instrument head of the surgical instrument is arranged in the protection chamber, and the handle, by which the surgeon operates the instrument, is arranged on the outside. A shaft of the instrument, arranged between the handle and the instrument head, forms a seal with the sealing surface surrounding the through-opening. The surgeon can guide the instrument from the outside during the treatment step. By means of the sealing surface, this can be done without aerosols or the like being able to escape in the area of the through-opening.

The partition of the protective hood can comprise a viewing surface, which lies opposite the operation field during correct use. A surgeon looking in the direction of the operation field sees this viewing panel of the protective hood. To allow the surgeon a view of the operation field, the viewing surface can be transparent, such that the surgeon is able to discern structures in the operation field through the viewing surface.

The viewing surface is preferably oriented substantially parallel to the support surface. The distance between the support surface and the viewing surface can be between 1 cm and 5 cm, preferably between 2 cm and 4 cm. If the distance is too small, the protective hood mists up from the inside, such that the surgeon no longer has a view. If the distance is too great, droplets form on the inner side of the viewing surface, which likewise obstruct the surgeon's view. In addition to or alternatively to the choice of the suitable distance, the viewing surface can be provided, on its inner side directed toward the protection chamber, with a coating that improves wettability. Droplets thus spread out widthwise, so that ideally a continuous film is obtained through which a good view is afforded. For example, the surface can be coated with a silicone adhesive.

If, after the development of a continuous film, further liquid lands on the inner side of the viewing surface, this can lead to droplet formation. The inner side of the viewing surface can be provided with a structure that promotes the formation of droplets. For example, this can be an elevation or depression on the inner side of the viewing surface, where the liquid draws together as a result of surface tension. In this way, it is possible to control the formation of droplets in such a way that the surgeon's view is obstructed only very slightly.

In a preferred embodiment, the structure extends as far as an edge of the viewing surface. A droplet can then move along the structure into an area lying outside the viewing surface. From there, the droplet can, for example, flow down the side wall, where it is no longer a problem. For example, the structure can be a rib, which rises from the inner side, or a groove, which is formed in the inner side.

It is advantageous if the protective hood is designed such that, during the surgical treatment step, substances can be sucked out of the protection chamber. For this purpose, the partition of the protective hood can be provided with a suction opening which is separate from the through-opening and through which a probe of a suction device can be inserted into the protection chamber. The suction opening can be provided with a sealing surface, which forms a seal with the probe of the suction device.

The viewing surface can be a substantially flat surface. This makes it easier to clean the inner side of the viewing surface again when contaminating material has settled on it. The optical properties of the viewing surface can be such that light passes through substantially rectilinearly. By way of the viewing surface, the surgeon then sees basically the same image of the operation field as if he were looking directly at the operation field without a protective hood. In an alternative embodiment, the viewing surface acts as an optical lens, such that the surgeon sees an enlarged image of the operation field.

The larger the viewing surface, the better the surgeon's view. The viewing surface preferably corresponds to at least 70%, more preferably at least 90%, more preferably at least 100% of the surface area of the opening enclosed by the support surface. If the viewing surface is projected into the plane of the opening, in a direction perpendicular to the opening, then the viewing surface overlaps the opening preferably by at least 70%, more preferably by at least 90%, more preferably by at least 100%. The openings enclosed by the support surface can, for example, have a surface area of between 5 cm² and 25 cm².

The through-opening for the surgical instrument can extend through the viewing surface. In order to obstruct the surgeon's view as little as possible, the through-opening can be arranged eccentrically with respect to the viewing surface. It is also possible that the through-opening is arranged in another area of the protective hood, such that the through-opening only partially overlaps the viewing surface or does not overlap it at all.

The through-opening can be surrounded by a nozzle that extends upward from the partition. The nozzle can extend from the partition into the interior and/or exterior. The nozzle can enclose an angle of at least 30°, preferably at least 60°, more preferably approximately 90°, with the partition. The sealing surface for the surgical instrument is preferably arranged on an inner side of the nozzle. To make the connection and separation of the surgical instrument easier, the sealing surface can have a cone shape. The cone widens preferably in the direction of the exterior.

The nozzle can have a hinged design, such that the orientation relative to the partition of the protective hood can be changed. The nozzle can be made elastic, such that it returns to a starting position when force is no longer exerted on the nozzle. The indication for the angle between the nozzle and the partition relates to the starting position. Proceeding from the starting position, the nozzle can preferably be inclined in each direction by at least 15°, more preferably by at least 30°.

The hinged characteristic can derive from the fact that the nozzle is made of an elastic material. In addition or alternatively to this, the hinge can be defined structurally, for example by the material being thinner in the area of the hinge, or by the hinge having an oblique surface inclined relative to the axis of the nozzle, which oblique surface extends about the circumference of the nozzle. By means of a hinged configuration of the nozzle, it is possible to move the instrument head of the surgical instrument relative to the operation field, without the protective hood changing its position.

The nozzle can have a concertina-like portion, such that the length of the nozzle can be changed. The concertina-like portion is preferably positioned such that the sealing surface for the surgical instrument can be moved toward the partition or moved away from the partition. The sealing surface can form the portion of the nozzle farthest from the partition. By means of a change of length of the nozzle, the instrument head can be moved toward or moved away from the operation field without the protective hood changing its position.

An inwardly projecting structure can be provided on the inner side of the nozzle and forms an undercut for a surgical instrument inserted into the nozzle. A matching structure of the instrument can engage behind the undercut, such that the instrument is connected to the protective hood by a form-fit engagement. The structure on the inner side of the nozzle can be a lip, for example, which extends over the circumference of the nozzle.

The nozzle can be provided with an outwardly protruding tab which makes it easier to bring the sealing surface into engagement with the surgical instrument or to separate the sealing surface from the surgical instrument.

The protective hood can have a grip portion arranged between the support surface and the viewing surface. The grip portion can extend as a circumferential surface around the protection chamber. For easy maneuverability, it is advantageous if the grip portion is oriented substantially perpendicularly with respect to the support surface. The grip portion is preferably spaced apart from the support surface. The distance can be, for example, between 0.5 cm and 4 cm, preferably between 1 cm and 3 cm.

The support surface can be arranged on a deformable buffer element. This permits a change of shape of the support surface, such that the support surface can lie flat on differently configured surfaces. The buffer element is preferably elastically deformable, such that it returns to a starting state when no force is exerted. The buffer element can be formed in the manner of a cushion, which in itself deforms.

A buffer element in the form of a flexible wall portion that bends under pressure is also possible. The support surface can likewise be formed as a flexible wall portion that has a different orientation than the buffer element. The buffer element can be oriented substantially perpendicularly with respect to the operation field and can extend in the circumferential direction around the protection chamber, while the support surface is directed toward the operation field. The end of the buffer element directed away from the operation field can adjoin the grip portion of the protective hood.

The support surface can merge inwardly and/or outwardly into a rounded area directed away from the operation field. This makes it easier to move the protective hood relative to the operation field. In a preferred embodiment, the support surface extends inward from the buffer element. At its inner end, the support surface can be extended upward. The upward transition is preferably formed as a rounded area, such that the opening through which the operation field is accessible is bordered by a rounded edge.

To be able to move the protective hood relative to the operation field, it is also advantageous if the support surface is provided with a coating that promotes sliding.

In a preferred embodiment, the protective hood is a one-piece injection molding. The material can be inherently elastic. If the material is elastic, the protective hood can have undercuts in the demolding direction of the injection molds. Despite the undercuts, the protective hood can be released from the injection mold by elastic deformation. The material can, for example, be a silicone material, which is preferably biocompatible.

If the injection molding comprises a buffer element, a grip portion and a viewing surface, the respectively desired properties can be achieved by means of variation of the chosen material thickness. For example, the material thickness of the buffer element, which is intended to be easily deformable, can be less than the material thickness of the grip portion. The material thickness of the grip portion can be chosen such that the grip portion is given sufficient stability. The material thickness of the viewing surface can likewise be greater than the material thickness of the buffer element, such that the viewing surface also contributes to the stability of the protective hood. The support surface in turn is intended to be easily deformable, for which reason the material thickness here is preferably less than in the grip portion.

The invention also relates to a system comprising such a protective hood and a surgical instrument. The surgical instrument comprises a shaft, of which the circumferential surface is designed to form a seal with the sealing surface of the through-opening. An instrument head arranged at the distal end of the shaft can be guided through the through-opening and introduced into the protection chamber of the protective hood. The insertion can take place under elastic deformation of the protective hood. The surgical instrument can have a suction device, of which the front end is inserted together with the instrument head into the protection chamber. The channel of the suction device can extend in the interior of the shaft of the surgical instrument.

The surgical instrument can be, for example, an ultrasonic surgical instrument in which the instrument head is oscillated, by an ultrasonic transducer, at a frequency in the ultrasonic range. It is also possible that the surgical instrument is, for example, a rapidly rotating drill or a milling cutter.

### BRIEF DESCRIPTION OF THE DRAWINGS

The invention is described below on the basis of advantageous illustrative embodiments and with reference to the attached drawings, in which:

FIG. 1 shows a protective hood with a surgical instrument;

FIG. 2 shows a view of a protective hood;

FIG. 3 shows the protective hood according to FIG. 2 from another perspective;

FIG. 4 shows the protective hood according to FIG. 2 in a view from below;

FIG. 5 shows the protective hood according to FIG. 2 in a view from above; and

FIG. 6 shows an alternative embodiment of a protective hood in a cross-sectional view.

### DETAILED DESCRIPTION

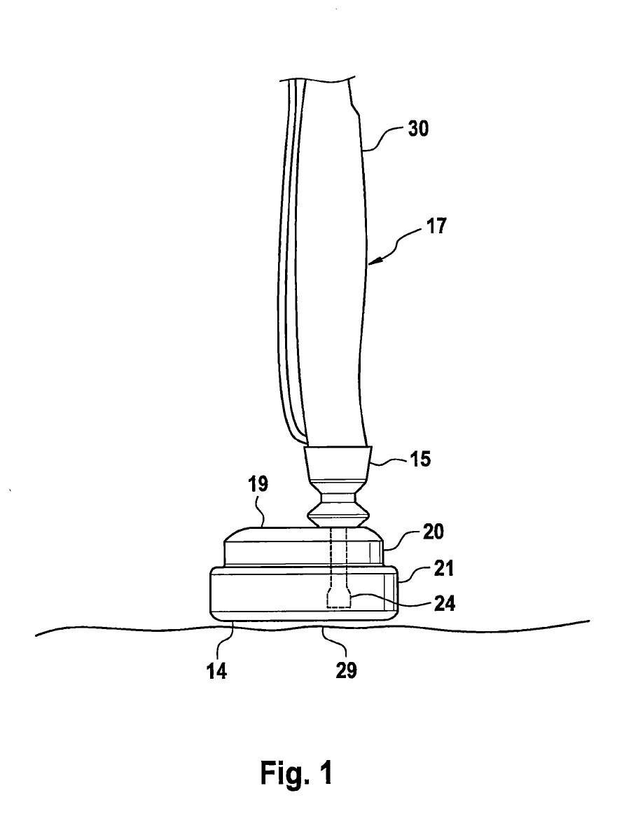

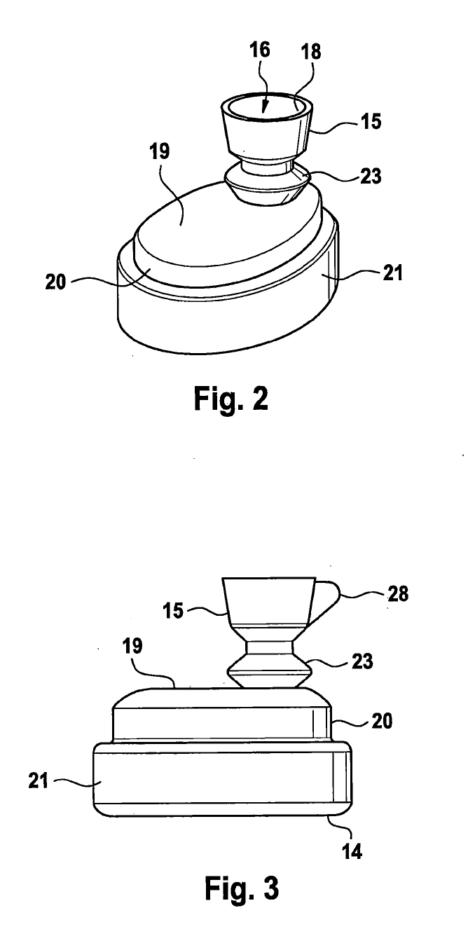

As shown in FIG. 1, a protective hood has, on its underside, a support surface 14 extending about an opening. The support surface 14 is designed to be placed onto the area around an operation field 29, such that the operation field is accessible through the opening. The opening is then closed by the body of the patient, such that a protection chamber forms in the interior of the protective hood, which protection chamber is separated from the exterior. The separating surface between the protection chamber and the exterior is composed of a viewing surface 19, a grip portion 20 and a buffer element 21.

A nozzle 15, which encloses a through-opening 16, is formed on the end of the protective hood lying opposite the support surface 14. The through-opening 16 extends from the exterior through the nozzle 15 into the protection chamber. FIG. 1 shows a surgical instrument 17, of which the shaft is inserted into the nozzle 15. An instrument head 24 of the surgical instrument 17 is arranged in the protection chamber, while the handle 30, by which the surgeon operates the instrument, is arranged on the outside. The shaft, which extends between the handle and the instrument head 24, is located in the through-opening 16. A circumferential surface of the shaft bears on an inner side of the nozzle 15 serving as a sealing surface 18, such that the transition from the shaft to the nozzle is sealed off.

Surgical treatment steps can thus be performed with the surgical instrument in the interior of the protection chamber, the instrument being operated entirely from the outside. Aerosols, vapors and the like, which arise during the surgical treatment step, are retained inside the protection chamber, and therefore contamination of the environment is avoided.

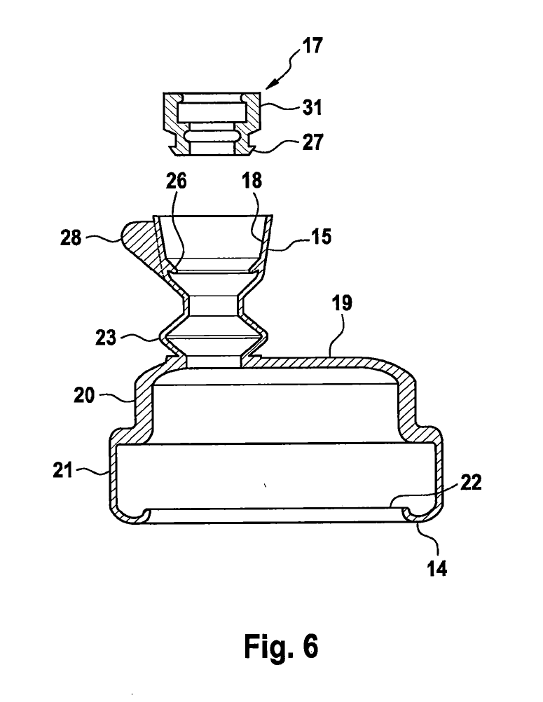

The protective hood is produced as a one-piece injection molding from a silicone material. As FIG. 6 shows, the protective hood has a plurality of undercuts in the vertical direction. On account of the flexibility of the silicone material, it is nonetheless possible to remove the injection mold in this direction.

The silicone material is transparent in the areas in which the protective hood is flat or approximately flat. A surgeon can therefore see through the material of the protective hood in these areas and discern structures in the operation field. The viewing surface 19 at which the surgeon looks is in particular designed as an approximately flat surface. The viewing surface 19 is arranged above the operation field, such that the surgeon can see the operation field through the viewing surface 19.

The through-opening 16 extends through the viewing surface in the edge area of said viewing surface 19. Beside the through-opening 16 and the nozzle 15 surrounding the through-opening 16, a large surface remains for looking through.

The horizontally oriented viewing surface 19 is adjoined by a substantially vertically oriented grip portion 20. The surgeon can take hold of the grip portion 20 in order to position the protective hood or move the protective hood relative to the operation field. As the cross-sectional view in FIG. 6 shows, the protective hood has a greater material thickness in the area of the viewing surface 19 and of the grip portion 20. The viewing surface 19 and the grip portion 20 thus together contribute to giving the protective hood sufficient stability.

The grip portion 20 is adjoined by a buffer element 21, which merges from a perpendicular portion with a rounding into the support surface 14. The material thickness in the area of the buffer element 21 and of the support surface 14 is less than in the grip portion 20. The buffer element 21 and the support surface 14 can thus be deformed with light pressure, as a result of which it is possible to adapt the protective hood to uneven surfaces in the area surrounding the operation field.

The inner end edge 22 of the support surface 14 is extended slightly upward. The support surface 14 merges with a rounded area into the end edge 22. Since the support surface 14 is bordered on both sides by a rounded area, this makes it easier to move the protective hood relative to the operation field.

Between the sealing surface 18, arranged at the upper end, and the viewing surface 19, the nozzle 15 comprises a concertina-like portion 23. The concertina-like portion 23 acts, on the one hand, as a joint for the nozzle 15, such that the nozzle can change its orientation relative to the protection chamber. For a surgical instrument inserted into the nozzle 15, this means that the instrument head 24 can be moved inside the protection chamber.

By means of the concertina-like portion 23, it is also possible for the length of the nozzle 15 to be changed, such that the instrument head can be moved up and down in the protection chamber.

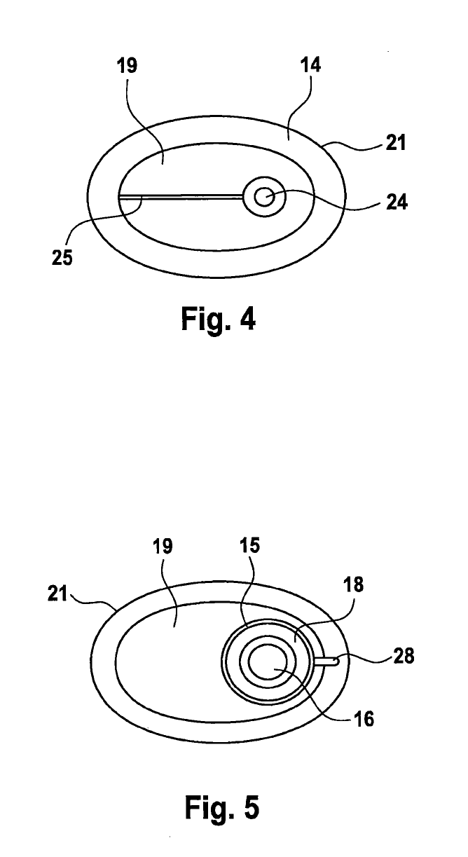

The opening shown in FIG. 4, and enclosed by the support surface 14, has a surface area of approximately 10 cm². According to FIG. 5, the viewing surface 19 has a shape and size similar to those of the opening. The support surface 14 has a width of approximately 5 mm. The distance between the support surface 14 and the viewing surface 19 corresponds substantially to the height of the protection chamber, being approximately 3 cm. In this way, the viewing surface 19 is sufficiently far away from the operation field to ensure that the inner side does not mist up. On the other hand, the viewing surface 19 is still sufficiently close to the operation field to ensure that the swirling liquid is able to form a continuous film on the inner side of the viewing surface 19. A protruding rib 25, shown in FIG. 4, is formed on the inner side of the viewing surface 19, on which protruding rib 25 it is possible for excess liquid to gather and form droplets. The droplets move along the rib 25 in the direction of the grip portion 20 and then flow downward, where they are no longer a problem.

In the embodiment in FIG. 6, a peripheral lip 26 is formed in the nozzle 15 at the lower end of the sealing surface 18. A peripheral projection 27 is arranged on the shaft 31 of the surgical instrument 17, which is shown only in part in FIG. 6. When the shaft 31 of the instrument 17 is inserted fully into the nozzle 15, the projection 27 engages behind the lip 26, such that the protective hood is connected to the instrument 17 by a form-fit engagement. In order to release the instrument 17 again from the protective hood, the elastic material of the nozzle 15 is pulled outward, such that the form-fit engagement is canceled. To make the release easier, a tab 28, by which the nozzle can be pulled, is formed on the outer side of the nozzle 15.

1. A protective hood for a surgical operation field, said protective hood having a partition, which separates a protection chamber from an exterior, and having a support surface surrounding the operation field, characterized in that the partition has a through-opening, and in that the through-opening is surrounded by a sealing surface for a shaft of a surgical instrument, and in that the support surface extends inward from a buffer element and is extended upward at its inner end.

2. The protective hood as claimed in claim 1, characterized in that the partition comprises a viewing surface made of a transparent material.

3. The protective hood as claimed in claim 2, characterized in that the viewing surface, on its inner side directed toward the protection chamber, is provided with a coating that improves wettability.

4. The protective hood as claimed in claim 2, characterized in that the inner side of the viewing surface is provided with an elevation or depression, which extends in particular as far as an edge of the viewing surface.

5. The protective hood as claimed in claim 1, characterized in that the protective hood is provided with a suction opening.

6. The protective hood as claimed in claim 1, characterized in that the viewing surface is designed as an optical lens.

7. The protective hood as claimed in claim 1, characterized in that the through-opening is arranged eccentrically with respect to the viewing surface.

8. The protective hood as claimed in claim 1, characterized in that the sealing surface is arranged on the inner side of a nozzle.

9. The protective hood as claimed in claim 8, characterized in that the orientation of the nozzle with respect to the partition and/or the length of the nozzle can be modified.

10. The protective hood as claimed in claim 8, characterized in that an inwardly projecting structure is provided on an inner side of the nozzle and forms an undercut for a surgical instrument inserted into the nozzle.

11. The protective hood as claimed in claim 1, characterized in that the support surface merges into a rounded area directed away from the operation field.

12. The protective hood as claimed in claim 1, characterized in that the support surface is provided with a coating that promotes sliding.

13. The protective hood as claimed in claim 1, characterized in that the protective hood is produced as a one-piece injection molding.

14. The protective hood as claimed in claim 13, characterized in that a buffer element and a grip portion are arranged between the support surface and the viewing surface, and in that the material thickness of the buffer element is less than the material thickness of the grip portion.

15. A system comprising a protective hood, as claimed in claim 1, and of a surgical instrument, characterized in that the instrument has a shaft having a circumferential surface arranged between a handle and an instrument head, the circumferential surface of which shaft is designed to form a seal with the sealing surface of the through-opening.

16. The protective hood as claimed in claim 3, characterized in that the inner side of the viewing surface is provided with an elevation or depression, which extends in particular as far as an edge of the viewing surface.

17. The protective hood as claimed in claim 2, characterized in that the protective hood is provided with a suction opening.

18. The protective hood as claimed in claim 3, characterized in that the protective hood is provided with a suction opening.

19. The protective hood as claimed in claim 4, characterized in that the protective hood is provided with a suction opening.

20. The protective hood as claimed in claim 2, characterized in that the viewing surface is designed as an optical lens.
